(19) United States
(12) Patent Application Publication
(10) Pub. No.: US 2004/0171518 A1
(43) Pub. Date: Sep. 2, 2004

(54) Compounds for protein stabilization and methods for their use

(75) Inventors: William Peter Van Antwerp, Valencia, CA (US); Joseph C. Walsh, Pacific Palisades, CA (US)

Correspondence Address: GATES & COOPER LLP, HOWARD HUGHES CENTER, 6701 CENTER DRIVE WEST, SUITE 1050, LOS ANGELES, CA, 90045, US

(73) Assignee: Medtronic MiniMed, Inc.

(21) Appl. No.: 10/375,383

(22) Filed: Feb. 27, 2003

(51) Int. Cl. A61K 38/28; A61K 31/495

(52) U.S. Cl. 514/3; 514/255.02

(57) ABSTRACT

The present invention is directed to stabilized polypeptide compositions. Typical embodiments of the present invention provide improved methods and materials for maintaining the stability of insulin polypeptide formulations. In particular, the disclosure provided herein teaches that the aggregation of insulin polypeptides can be inhibited by combining them with a class of compounds having the general formula A¹—L¹—S—L²—A², wherein S comprises from one to seven consecutive atoms selected from the group consisting of nitrogen, carbon, and oxygen, wherein at least one of the atoms is a carbon atom; L¹ and L² are linking groups having from two to twelve atoms selected from the group consisting of nitrogen, carbon, oxygen, sulfur, and phosphorus; and A¹ and A² are carboxylic acid groups.

### RELATED APPLICATIONS

This application is related to U.S. application Ser. No. 09/344,676, filed Jun. 25, 1999, and U.S. application Ser. No. 10/080,034, filed Feb. 21, 2002, the contents of each of which are incorporated herein by reference.

### BACKGROUND OF THE INVENTION

1. Field of the Invention

The present invention relates generally to methods of stabilizing pharmaceutical compositions and to improved formulations for use with therapeutic polypeptides.

2. Description of Related Art

In continuous infusion systems, a fluid containing a therapeutic agent is pumped from a reservoir, usually to a subcutaneous, intravenous, or intraperitoneal tissue space. The reservoir, which is refilled periodically, is attached to the patient's body, or is implanted in the patient's body. In either case, the patient's body heat and body motion, plus turbulence in the tubing and pump impart a relatively high amount of thermo-mechanical energy to the formulation. In the interest of minimizing the frequency with which the reservoir is refilled, and of minimizing the size of the reservoir, stable formulations having a relatively high concentration of the therapeutic agent are highly advantageous. Consequently, stable formulations of therapeutic agents are particularly important for use in delivery devices that expose these agents to elevated temperatures and/or mechanical stress.

A typical context for such continuous infusion systems involves the treatment of diabetes and related syndromes by the administration of insulin and its analogs. Stable insulin formulations, for example, are required for use in continuous infusion systems and related devices. Insulin formulations for implantable pump use preferably possess several characteristics including exceptional physical and chemical stability. Commercial insulin preparations are typically designed to have a stable shelf life of 1 to 2 years when stored at refrigerator temperatures in glass vials or cartridges. In actual use however, insulin is typically used in a syringe (for immediate injection) in an insulin pump (for up to a week) or in an insulin pen (for a week or two) or in an implantable pump for up to 90 days.

Optimized formulations of insulin for use in continuous infusion systems should remain soluble and substantially free of aggregation, even though subjected to the patient's body heat and motion for periods ranging from a few days to several months. In this context, instability is promoted by the higher insulin concentrations that are desirable for continuous infusion systems and by the thermo-mechanical stress to which formulations are exposed in continuous infusion systems. Therefore, improvement in the physical and chemical stability of concentrated insulin formulations is urgently needed to facilitate their use in continuous infusion systems. In particular, insulin formulations for implantable pump use should possess chemical and physical stability in the harsh environment of the implantable pump. In addition, the formulations should be stable in a glass cartridge or vial during long term storage and should be stable for at least 90 days at physiological temperatures, all while being constantly agitated inside a metal container (typically titanium) having a relatively hydrophobic surface (e.g. TiO₂ with an average contact angle of more than 60°).

The development of insulin analogs for the treatment of diabetes provides new formulations for use in continuous infusion devices. For example, rapid-acting insulins, known as monomeric insulin analogs, are well-known in the art, and are disclosed in Chance, et al. U.S. Pat. No. 5,514,646, issued May 7, 1996; Brems, et al., Protein Engineering, 6:527-533 (1992); Brange, et al., EPO publication No. 214,826 (published Mar. 18, 1987); and Brange, et al., Current Opinion in Structural Biology 1:934-940 (1991). Monomeric insulin analogs are absorbed much faster than insulin, and are ideally suited for postprandial control of blood glucose levels in patients in need thereof. They are also especially well-suited for administration by continuous infusion for both prandial and basal control of blood glucose levels because of their rapid absorption from the site of administration. Unfortunately, monomeric insulin analog formulations have a propensity to aggregate and to become unstable when exposed to thermo-mechanical stress. Aggregation can arise when histidines or hydrophobic amino acids in proteins are exposed to environment which results in the proteins denaturing to form β pleated sheets. Aggregation may manifest in a variety of phenomena including the precipitation of higher-order insulin species. Aggregation is a significant problem with such formulations and can prevent reproducible delivery of effective therapeutic doses of monomeric insulin analogs, and may also cause irritation at the administration site or even an enhanced systemic immunological response. Consequently, insulin polypeptides stabilized against aggregation are highly desirable.

While a number of aqueous formulations which stabilize polypeptide compositions have been identified in the art, the destabilization of polypeptides in solution (particularly insulin polypeptides) due to processes such a polypeptide aggregation continues to create problems for medical practitioners. Consequently, there is a need for new methods and compositions which overcome the problems of the prior art. This need is fulfilled by the invention that is described below.

### SUMMARY OF THE INVENTION

The invention described herein has a number of embodiments. Typical embodiments of the invention pertain to compounds having the general formula A¹—L¹—S—L²—A² wherein S is a molecular scaffold moiety comprising from one to seven atoms that are consecutively linked and selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are organic acid moieties. In particular, the disclosure provided herein teaches that these compounds can be used methods to stabilize polypeptide formulations by inhibiting the aggregation of the polypeptides therein.

The invention disclosed herein has a number of embodiments. A preferred embodiment of the invention is a method of inhibiting the aggregation of insulin polypeptides in a solution comprising combining the insulin polypeptides with a compound having the general formula: A¹—L¹—S—L²—A² wherein S is a molecular scaffold which comprises from one to seven consecutively linked atoms selected from the group consisting of nitrogen, carbon, and oxygen, wherein at least one of the atoms is a carbon atom, L¹ and L² are linking groups having from two to twelve consecutively linked atoms selected from the group consisting of nitrogen, carbon, oxygen, sulfur, and phosphorus; and A¹ and A² are organic acid groups. In this method, the amount of compound combined with the insulin polypeptides is sufficient to inhibit the aggregation of the insulin polypeptides in the solution. Skilled artisans will understand that a variety of permutations of this formula can be generated and used in the methods disclosed herein. For example, the compound can be constructed to further comprise additional groups such as phenolic moieties, groups which are known in the art to further stabilize insulin polypeptides.

A related embodiment of the invention is a method of increasing the stability of insulin polypeptides in a solution comprising combining the insulin polypeptides with a compound having the general formula A¹—L¹—S—L²—A², wherein the amount of compound in the solution is sufficient to inhibit the aggregation of the insulin polypeptides in the solution. Another related embodiment of the invention is method of producing a stabilized insulin solution comprising combining insulin with a compound having the general formula A¹—L¹—S—L²—A², wherein the compound inhibits the aggregation of the insulin in the solution. In preferred embodiments of the invention the polypeptide is LISPRO insulin and the compound has a general formula shown in FIG. 2.

Another embodiment of the invention is a method of making a compound for use in inhibiting the aggregation of insulin polypeptides in solution comprising a first step of constructing a compound having the general formula A¹—L¹—S—L²—A², wherein S comprises a molecular scaffold designed to have from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are organic acid groups. Once this compound is constructed, the second step of the method entails testing the compound in an assay of insulin polypeptide aggregation, wherein the level of insulin polypeptide aggregation that is observed in a solution comprising insulin polypeptides and the compound is less than that observed in a control sample to which no compound has been added. A related embodiment of the invention is a method of identifying a compound for use in inhibiting the aggregation of insulin polypeptides in solution comprising combining insulin polypeptides with a compound having the general formula A¹—L¹—S—L²—A² and then characterizing the compound in an assay of insulin polypeptide aggregation, wherein the level of insulin polypeptide aggregation that is observed in the solution comprising the insulin polypeptides and the compound is less than that observed in a control sample to which no compound has been added. In such methods, the inhibition of aggregation is typically observed in a spectrophotometric assay of turbidity or a Thioflavin-T assay (e.g. as described in Example 1 below) or laser-induced light scattering.

The formulations of the invention disclosed herein have a number of preferred embodiments. For example, because monomeric insulin analog formulations have a propensity to aggregate and to become unstable when exposed to thermo-mechanical stress, the methods and formulations described herein are particularly useful for addressing this characteristic of these analogs. For example, in one such preferred embodiment, the insulin polypeptide is a monomeric insulin analog formulations such as LISPRO insulin. As is described herein, the various formulations of the invention can comprise a wide variety of molecules typically used in this art. For example, certain preferred embodiments of the invention are those in which the formulation includes zinc. In addition, the various formulations are preferably pharmaceutical compositions (e.g. prepared in a pharmaceutically acceptable carrier).

The aggregation of polypeptides in pharmaceutical compositions may enhance undesirable systemic immunological responses to antigenic sites present in the aggregated complex. Consequently, yet another embodiment of the invention is a method of inhibiting the generation of an immune response to exogenous insulin comprising administering the exogenous insulin to a subject in a solution in combination with a compound having the general formula A¹—L¹—S—L²—A² wherein S comprises from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are organic acid groups, wherein the amount of compound combined with the insulin polypeptides is sufficient to inhibit the aggregation of the insulin polypeptides in the solution. A related embodiment of the invention is a method of inhibiting the formation of immunoreactive insulin complexes in a solution comprising combining insulin in a solution a compound having the general formula A¹—L¹—S—L²—A², wherein the amount of compound combined with the insulin polypeptides is sufficient to inhibit the aggregation of the insulin polypeptides in the solution.

For use in the applications described or suggested above, kits are also provided.

### BRIEF DESCRIPTION OF THE DRAWINGS

FIG. 1 provides a schematic of a mechanism of insulin aggregation.

FIGS. 2A and 2B provide illustrative examples of preferred compounds having the general formula: A¹—L¹—S—L²—A².

FIGS. 3A and 3B provide illustrative synthesis schemes for the production of compounds having the general formula: A¹—L¹—S—L²—A².

### DETAILED DESCRIPTION OF THE INVENTION

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted. For purposes of the present invention, as disclosed and claimed herein, the following terms and abbreviations have the following meanings.

As used herein, the terms “stable” “stabilized” and “stability” are used according to their broadest meaning and refer to the physical and chemical and/or biological stability of formulations of polypeptides such as insulin species. Instability in a protein formulation typically results in a decrease in the biological activity of the formulation over time. Loss of biological activity may be caused, for example, by the aggregation of the protein molecules which can form higher order polymers or even precipitates. For example, insulin has a propensity to aggregate when exposed to thermo-mechanical stress. A “stable” or “stabilized” formulation is one wherein a loss of biological activity associated with the instability of a formulation is reduced. As is known in the art, the relative stability of a formulation can be assessed, for example, by comparative analyses of the properties of a control formulation (e.g. one consisting of insulin polypeptides in the absence of the compounds disclosed herein having the general formula A¹—L¹—S—L²A² with a formulation under investigation (e.g. one consisting of insulin polypeptides in the presence of the compounds disclosed herein having the general formula A¹—L¹—S—L²—A². Stability, such as physical stability may be assessed by methods well-known in the art, including measurement of a sample's apparent attenuation of light (absorbance, or optical density). Such a measurement of light attenuation relates to the turbidity of a formulation. Turbidity is produced by aggregation or precipitation of proteins or complexes in the formulation. Other methods for assessing physical stability are well-known in the art.

The various forms of the verb “to aggregate” refer to a process whereby individual molecules or complexes associate to form aggregates. An exemplary aggregate is a polymeric assembly having molecules or complexes of monomeric insulin analog. Monomeric insulin analogs, and hexamer complexes thereof, have a propensity to aggregate when, for example, they are exposed to thermo-mechanical stress. Aggregation can proceed to the extent that a visible precipitate is formed.

The term “complex” means a composition having two or more parts, such as a compound in which a transition metal is coordinated to at least one ligand. Ligands include nitrogen-containing molecules, such as proteins, peptides, amino acids, and TRIS, among many other compounds. Monomeric insulin analog can be a ligand of divalent zinc ions.

The terms “polypeptide” and “protein” are used interchangeably herein and encompass natural, synthetic and recombinant polypeptides (e.g. recombinant human insulin and analogs thereof) having a desired biological activity, including polypeptides and proteins having deleted, replaced or altered amino acid sequences in comparison with the full-length natural polypeptide or biologically active fragments thereof.

The term “analog” refers to a molecule that is structurally similar or shares similar or corresponding attributes with another molecule. The term “homolog” refers to a molecule which exhibits homology to another molecule, by for example, having sequences of chemical residues that are the same or similar at corresponding positions. For example, insulin LISPRO is an analog of human insulin where the B28 Proline and B29 Lysine are interchanged. The effects of this change are to fundamentally decrease the propensity to form the hexameric insulin structure and to increase the relative amount of insulin monomer present in solution. DeFelippis and colleagues in U.S. Pat. No. 6,034,054 describe a formulation of LISPRO insulin analog that shows remarkable physical stability when measured in-vitro. The term “variant” refers to a molecule that exhibits a variation from a described type or norm, such as a protein that has one or more different amino acid residues in the corresponding position(s) of a specifically described protein (e.g. LISPRO insulin is a variant of human insulin). Those skilled in the art understand that the terms analog, homolog and variant are not mutually exclusive and that various molecules can meet more than one of these definitions.

The term “species” such as in “insulin species” is used according to its art accepted meaning and refers to those mammalian insulin proteins having a biological activity that allows them to be used in the treatment of diabetes such as human insulin and insulins from non-human mammals as well as variants of human insulin (e.g. porcine insulin and LISPRO insulin). Non-human insulin species generally share at least about 90% or more amino acid homology with human insulin (e.g. using BLAST criteria). The structure of human insulin is disclosed in Nature 187,483 (1960). A review of the research, development, and recombinant production of human insulin is found in Science 219, 632-637 (1983). See also U.S. Pat. Nos. 4,652,525 (rat insulin) and 4,431,740 (human insulin).

The terms “monomeric human insulin analog”, “monomeric insulin analog” and “human insulin analog” are well-known in the art, and refer generally to fast acting analogs of insulin (typically human insulin), which include: human insulin, wherein Pro at position B28 is substituted with Asp, Lys, Leu, Val, or Ala, and wherein position B29 is Lys or is substituted with Pro; AlaB26-human insulin, des(B28-B30) human insulin; and des(B27) human insulin. Such monomeric insulin analogs are disclosed in U.S. Pat. No. 5,514,646, WO 99/64598, WO 99/6459A2 and WO 96/10417A1.

The term “phenolic preservative” as used herein, typically refers to art accepted phenolic preservatives such as chlorocresol, m-cresol, phenol, or mixtures thereof.

An “isotonicity agent” is a compound that is physiologically tolerated and imparts a suitable tonicity to a formulation to prevent the net flow of water across cell membranes that are in contact with the formulation. Compounds such as glycerin are commonly used for such purposes at known concentrations. Other possible isotonicity agents include salts, e.g., sodium chloride, dextrose, and lactose.

The term “administer” means to introduce formulation of the present invention into the body of a patient in need thereof to treat a disease or condition.

The term “continuous infusion system” refers to a device for continuously administering a fluid to a patient parenterally for an extended period of time or for, intermittently administering a fluid to a patient parenterally over an extended period of time without having to establish a new site of administration each time the fluid is administered. The fluid contains a therapeutic agent or agents. The device has a reservoir for storing the fluid before it is infused, a pump, a catheter, or other tubing for connecting the reservoir to the administration site via the pump, and control elements to regulate the pump. The device may be constructed for implantation, usually subcutaneously. In such a case, the insulin reservoir will usually be adapted for percutaneous refilling. Obviously, when the device is implanted, the contents of the reservoir will be at body temperature, and subject to the patient's body motion.

The term “treating” refers to the management and care of a patient having a pathology such as diabetes or hyperglycemia, or other condition for which insulin (or other polypeptide) administration is indicated for the purpose of combating or alleviating symptoms and complications of those conditions. Treating includes administering a formulation of present invention to prevent the onset of the symptoms or complications, alleviating the symptoms or complications, or eliminating the disease, condition, or disorder.

### Characterization of the Invention

Embodiments of the present invention provide improved methods and materials for maintaining the stability of insulin formulations by inhibiting the aggregation of the insulin polypeptides. In particular, as disclosed herein, a class of compounds having the general formula A¹—L¹—S—L²—A² are shown to stabilize proteins such as insulin by inhibiting the aggregation of these polypeptides. In this structure, S comprises from one to seven consecutively linked atoms (when this number is greater than 2) selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are carboxylic acid groups.

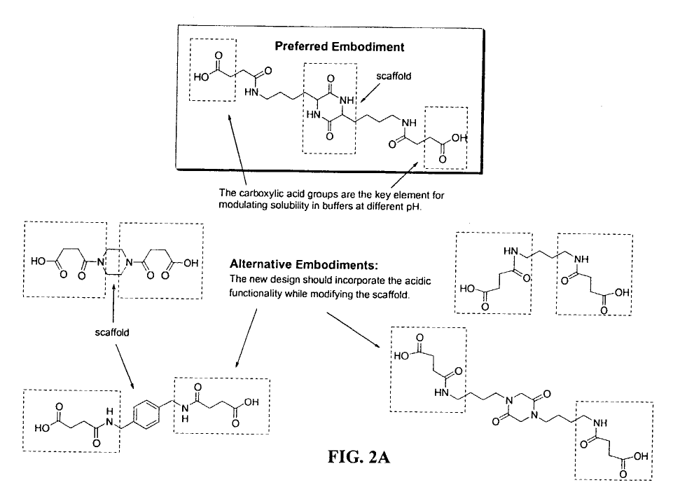

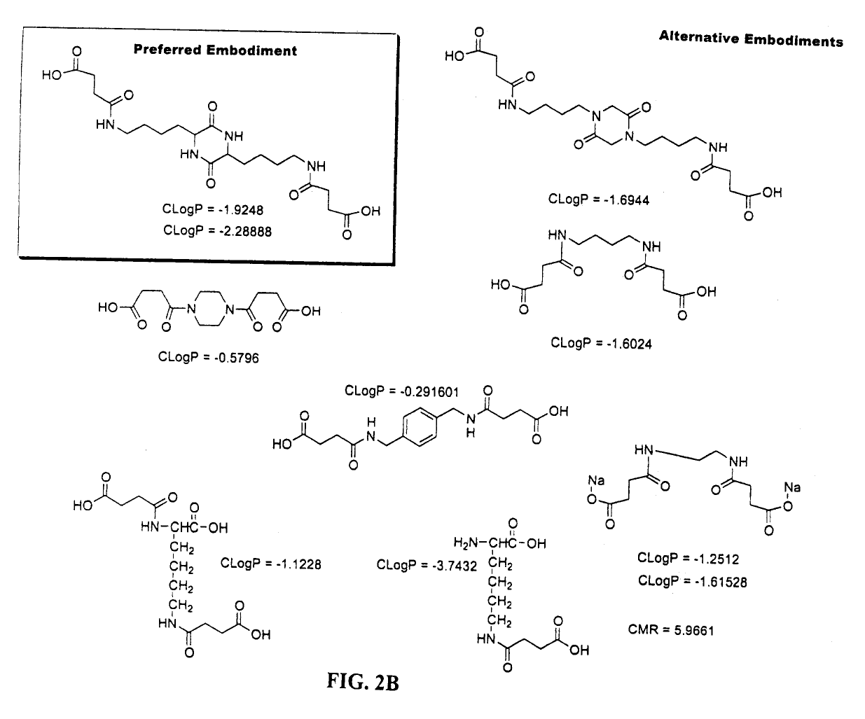

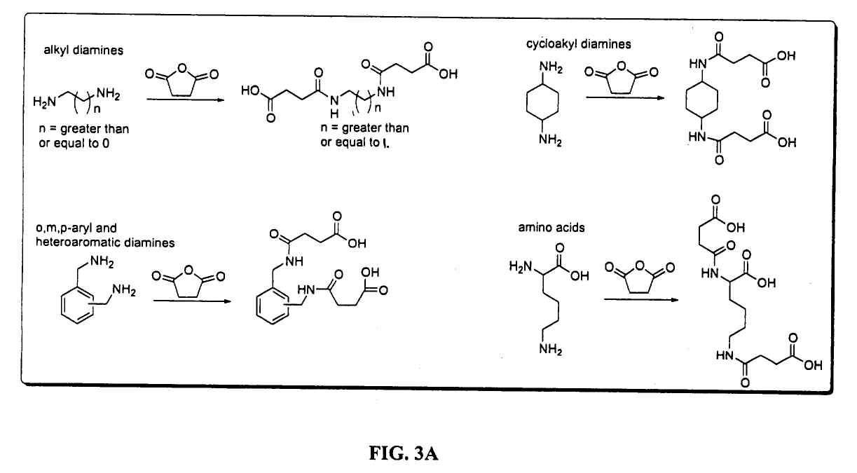

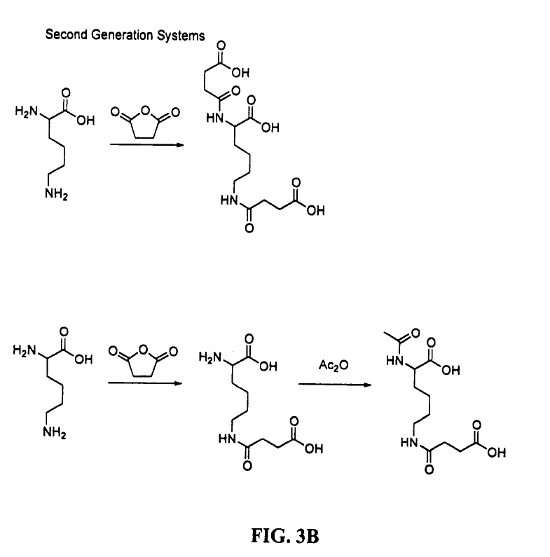

In a demonstration of the ability of stabilize proteins such as insulin by inhibiting the aggregation of these polypeptides with an insulin U400 formulation; the addition of such compounds improved the stability of the formulation by approximately 3-fold. Consequently such polypeptides are shown to be stabilized by combining them in solution with compound having the general formula A¹—L¹—S—L²—A² such as those shown in FIGS. 2 and 3. CD data provides evidence that these nitrogen compounds are reversibly interacting with monomer as well as insulin hexamers. While not being bound by a specific theory, it is possible that these types of compounds are inhibiting denaturation or capturing and stabilizing after partial denaturation.

In addition to keeping more of the proteins in their bioactive form, this inhibition of aggregation will reduce patient's immunological responses (humoral) to the protein (immune response is primarily directed to protein aggregates; see, e.g., JeanDidier et al., Diabetologia. 1995; 38(5):577-84). This is significant for example because for inhalable insulin products a significant issue is the immune response due to the reaction of the body with aggregated insulin. Therefore, a goal of medical researchers is to increase protein stability in order to reduce and/or or eliminate the undesirable immune response that is observed with the use of such formulations. While insulin polypeptides are the preferred polypeptides for stabilization due to their wide use as therapeutics, a decrease in the aggregation of a wide variety of other proteins is also contemplated. For example, other typical proteins to which the inventive methods can be applied are GLP, interleukin II, hGH, calcitonin and interferons.

As disclosed herein, the present invention pertains to methods for utilizing a class of compounds having the general formula A¹—L¹—S—L²—A² as well as certain of these compounds themselves. In a simplified model, such molecules can be described as a molecular scaffold with pH adjustment arms. In this context, the invention encompasses various compounds as long as they keep pH adjustment arms (scaffold can be manipulated) in a manner that preserves their functional attributes. In this context A (e.g. A¹ and A² is generally an organic acid and the inventive molecules can include mixed A groups or, alternatively, can be symmetrical as are compounds shown in FIGS. 2A and 2B.

Certain formulations containing the compounds disclosed herein (having the general formula A¹—L¹—S—L²—A²) have a number of preferred characteristics. As disclosed herein, polypeptides such as insulin associate with compounds having the general formula A¹—L¹—S—L²—A² in both soluble and insoluble contexts. In one preferred embodiment, the protein-compound system (e.g. the aggregate in solid phase, preferably insulin polypeptides associated with a compound shown in FIG. 2A) are in insoluble form a pH 4-6 but in a soluble form at pH 7.2. In another preferred embodiment, the protein-compound system is soluble at pH greater than 7 and insoluble at pH less than 6.5. In another preferred embodiment, the protein-compound system is insoluble at pH greater than 8 most preferably greater than 10. Typically the compounds have an insulin affinity of about 5-10% of weight. Understandably such formulations are design to exhibit no significant toxicological or bioactivity issues using one of the many assays for such activities known in the art. In addition, in certain particulate formulations of the invention the preferred particle diameter is less than about 2 μm (Mean Aerodynamic Diameter). As certain embodiments of the invention described herein pertain to insulin polypeptides encapsulated in the compounds disclosed herein, this allows artisans to control certain aspects of the polypeptide monomers via encapsulation technology.

The advantages of various embodiments of the invention are clarified when one considers problems associated with the instability of insulin formulations. The chemical stability of insulin is primarily governed by two main reactions, both of which are temperature dependent. A first destabilizing reaction that occurs in more acidic conditions is the deamidation of the A²¹ and B³ positions. A second set of destabilizing reactions occur at relatively higher pH values and involve changes in the disulfide linkages that define the structure of the insulin molecule. Insulin and its analogs contain three disulfide bonds, two between the A and B chains and one that joins two portions of the A chain. At relatively higher pH values these disulfide linkages can be broken and occasionally scrambled. The molecules that result from disulfide breaking have no biological activity and can lead to further aggregation of insulin in an autocatalytic reaction. The physical stability of insulin is primarily governed by two major factors, the presence of any insulin molecules that have had disulfide linkages disrupted or by insulin monomers that have been denatured by contact with hydrophobic surfaces. Physical instability is manifested as aggregation of the insulin (turbidity) and the generation of high molecular weight insulin polymers. Neither the aggregates nor the polymers have insulin biological activity and certain studies in the art indicate that insulin aggregates are potential causes of elevated levels of anti-insulin antibodies.

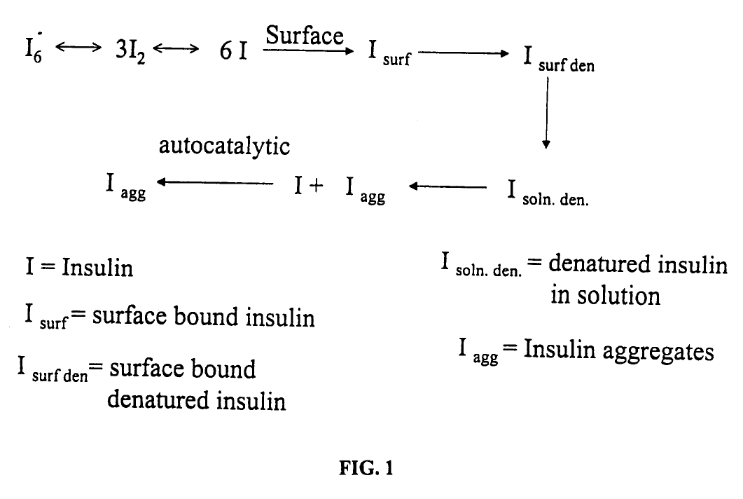

Insulin chemical stability is predominately thermally driven, with no large differences between chemical stability in the pump and in the glass primary packaging. Physical stability however is strongly dependent on both the formulation and the materials and shapes of the materials that are in contact with the insulin. In 1997, a model of insulin/pump interactions was published that showed the importance of an interaction between the free insulin monomer in the formulation with the surface of the pump (Proceedings of AIDSPIT (Artificial Insulin Delivery Systems, Pancreas and Islet Transplant) meeting, February, 1997). The model is illustrated in the equation shown in FIG. 1 which is summarized below:

I₆ ←>3I₂←>6I→I_{surf}→I_{surf den}→I_{sol den}→I_{agg}+I→I_{agg} autocatalytic

This equation says that insulin in solution in a container such as a bottle or pump is an equilibrium between the insulin hexamer, dimer and monomer. Insulin monomer can deposit on a surface to make I_{surf}. On the surface, insulin can denature to make I_{surf den}. Denatured insulin can then fall off the surface to make I_{sol den}, denatured insulin in solution. This insulin is the seed for aggregation and once aggregates form, even soluble aggregates; they react in an autocatalytic manner with insulin monomer to form further aggregates. This model provides insight into methods of stabilizing insulin that allow the skilled artisan to make a variety of stabilized insulin formulations. For example, by reducing the amount of monomeric insulin in an insulin composition, the overall aggregation and destabilization of the insulin molecules in the solution is reduced.

Without being bound by a specific theory, by using data pertaining to insulin instability, embodiments of the invention disclosed herein address the above noted problems by identifying formulation conditions which enhance insulin stability thereby facilitating its use, particularly in pump infusion systems. Specifically, the observation that a class of compounds having the general formula A¹—L¹—S—L²—A² are shown to stabilize proteins such as insulin by inhibiting monomer aggregation allows the use of such compound in a variety of formulations and methods designed to stabilize insulin formulations.

The formulation disclosure provided herein enhances the therapeutic delivery of biologically active insulin in a number of contexts. For example, it is well known that some patients who use programmable external insulin pumps and insulin LISPRO (CSII, Continuous Subcutaneous Insulin Infusion) suffer from premature site loss. Thus site loss manifests itself by unexplained hyperglycemia in the absence of any discernable pump malfunction. Typically, patients using CSII will program a meal bolus appropriate for the meal and have an unexplained hyperglycemia several hours later. In most cases, the pump actually delivers the appropriate amount of insulin from the pump reservoir, but no concomitant blood glucose diminution occurs. There are several plausible explanations for these phenomena including a significant loss of insulin potency in the pump reservoir, anti-insulin antibodies, insulin degradation at the tip of the subcutaneous infusion set and others. The cause of site loss is likely to involve a biological response to infusion of denatured or partially denatured insulin monomer. Further, local cellular responses to unfolded proteins are likely to evoke a localized production of non-specific protease activity.

The insulin formulations of the present invention are specifically designed to address problems in the art related to the destabilization of insulin species, a phenomena which can be measured by a number of procedures known in the art.

Interestingly, certain molecules that are related to the aggregation inhibiting compounds disclosed herein appear to have a number of different uses in related contexts. Such molecules are, for example, used in procedures involving the removal of zinc from various therapeutic formulations as well as to facilitate the delivery of molecules such as antigens, where the molecule is intended to elicit a protective immune response (see, e.g. U.S. Pat. Nos. 6,071,497 and 6,444,226 which are incorporated herein by reference). However, the art that associated with these different uses in different contexts teaches that the side chains of molecules used to remove zinc from therapeutic formulations or facilitate the delivery of molecules such as antigens can contain also basic side groups or combinations of basic and acidic side groups. The significant structural and functional differences between molecules having only acidic side groups such as the aggregation inhibiting molecules disclosed herein (e.g. those shown in FIGS. 2A and 2B) as compared molecules that can contain basic side groups or combinations of basic and acidic side groups (e.g. those described U.S. Pat. Nos. 6,071,497 and 6,444,226) suggests that different mechanisms are involved the various methods which employ this class of molecules. As indicated below, assays such as the Thioflavin-t assay disclosed in Example 1 below are provided in order to facilitate the identification of those molecules which function in the methods for inhibiting the aggregation of polypeptides.

### Typical Embodiments of the Invention

As described herein, the inclusion of a class of compounds having the general formula A¹—L¹—S—L²—A² stabilizes proteins such as insulin by inhibiting monomer aggregation. Consequently, the formulations provided herein include insulin polypeptides combined with compounds having the general formula A¹—L¹—S—L²—A² in an amount sufficient to inhibit aggregation. The basis for the insulin components of the mixtures described herein can be found in art describing typical insulin formulations (e.g. human porcine and bovine insulin as well as insulin analogs such as LISPRO insulin).

The invention disclosed herein has a number of embodiments. A preferred embodiment of the invention is a method of inhibiting the aggregation of polypeptides in a solution comprising combining the polypeptides with a compound having the general formula A¹—L¹—S—L²—A². Representative molecules having this formula are shown in FIGS. 2 and 3. In preferred embodiments of the invention the polypeptide is LISPRO insulin and the compound has a general formula shown in FIG. 2. In such methods, the amount of compound combined with the polypeptides is sufficient to inhibit the aggregation of the polypeptides in the solution.

In the general formula A¹—L¹—S—L²—A², S is a molecular scaffold moiety comprising from one to seven consecutively linked atoms selected from the group consisting of nitrogen, carbon, and oxygen, wherein at least one of the atoms is a carbon atom. In a representative embodiment, this molecular scaffold moiety is cyclic in structure, preferably in the form of a six membered ring (e.g. has a structure analogous to cyclohexane or benzene molecules). In certain preferred embodiments of this molecular scaffold moiety, one or two of the constituent members of the six membered ring are nitrogen atoms with the remaining constituent members being carbon atoms. Alternatively, this molecular scaffold moiety is linear and comprises 1, 2, 3, 4, 5, 6 or 7 carbon atoms. As noted above, such linear molecular scaffold moieties can further comprise one or more nitrogen and/or oxygen atoms. In addition, in this formula; L¹ and L² are linking groups having from one to twelve consecutively linked atoms selected from the group consisting of nitrogen, carbon, oxygen, sulfur, and phosphorus. In a representative embodiment, one or both of the linking groups is in the form of linear chain of 1-12 atoms (and is preferably composed primarily of carbon atoms). In certain preferred embodiments, one or more of the linear chain of 1-12 consecutively linked atoms ate nitrogen and/or oxygen atoms. In addition, in this formula A¹ and A² ate carboxylic acid groups (including functional derivatives thereof) having a structure that is analogous to formic, acetic, propionic acids etc. (see, e.g. ORGANIC CHEMISTRY (Robert T. Morrison and Robert N. Boyd eds., 6^{th} ed. 1992), pages 713-796 which are incorporated herein by reference). In certain preferred embodiments of the invention, A¹ and A² are relatively small groups having less than 20 atoms. In addition, as illustrated above, in certain embodiments of the invention, the molecular scaffold moiety, the linker moieties and/or the carboxylic acid moieties are comprised a subset of the total possible atomic constituents identified above, for example only nitrogen, carbon, and oxygen.

A specific formula for highly preferred compound of the invention having the general formula A¹—L¹—S—L²—A² is shown at the top of FIG. 2A. In such highly preferred embodiments, S comprises 6 consecutively linked atoms (e.g. four carbon atoms and two nitrogen atoms) that are linked to form a hexameric ring structure. In such highly preferred embodiments, L¹ and L² each consist of eight consecutively linked atoms (e.g. seven carbon atoms and one nitrogen atom) and A¹ and A² each consist of small carboxylic acid groups (e.g. having one carbon atom, two oxygen atoms and one hydrogen atom). In characterizing the substituent groups of such compounds, skilled artisans further understand that because the atoms that form the subunits of the formula A¹—L¹—S—L²—A² are inextricably linked, there may be a certain amount of overlap as for example, one characterizes a specific portion of the molecules as being either a part of the linking groups or a part of the carboxylic acid groups etc. In this context, skilled artisans understand that this overlap is taken into account by the clearly defined parameters for the individual substituent groups that are provided herein.

Skilled artisans understand that one can easily manipulate the structure A¹—L¹—S—L²—A² to create a variety of compounds which maintain this essential constituent structure and can be used in the described methods of inhibiting the aggregation of polypeptides in a solution. For example, while the atoms that are typically covalently bound to the essential constituent atoms described above such as the consecutively linked atoms of the molecular scaffold (or the linker arms etc.) are typically hydrogen and oxygen as shown in FIG. 2, their substitution with like chemical moieties is within the skill of those in the art (e.g. a compound in which a hydrogen atom on a consecutively linked atom of the molecular scaffold or the linker arms etc. is substituted with a methyl group, an amino group, or an ethyl group etc.). In certain preferred embodiments of the invention, the compound can be constructed to include a phenolic moiety, a group known in the art to further stabilize insulin polypeptides (see, e.g. Tang et al., Biochemistry 1999 Sep. 14;38(37):12041-51). As disclosed herein, the extent to which any such species of compound having the formula A¹—L¹—S—L²—A² can inhibit the aggregation of polypeptides is readily observed in one of the assays for aggregation known in the art, for example a spectrophotometric assay of turbidity, a Thioflavin-T assay (e.g. as described in Example 1 below) or laser-induced light scattering.

A preferred embodiment of the invention is a method of inhibiting the aggregation of insulin polypeptides in a solution comprising combining the insulin polypeptides with a compound having the general formula: A¹—L¹—S—L²—A² wherein S is a molecular scaffold which comprises from one to seven atoms selected from the group consisting of nitrogen, carbon, and oxygen, wherein at least one of the atoms is a carbon atom, L¹ and L² are linking groups having from two to twelve consecutively linked atoms selected from the group consisting of nitrogen, carbon, oxygen, sulfur, and phosphorus; and A¹ and A² are carboxylic acid groups. In this method, the amount of compound combined with the insulin polypeptides is sufficient to inhibit the aggregation of the insulin polypeptides in the solution. As noted above, skilled artisans will understand that a variety of permutations of this formula can be generated and used in the methods disclosed herein. In an exemplary embodiment, the compound can be constructed to include a phenolic moiety, a group known in the art to further stabilize insulin polypeptides (see, e.g. Tang et al., Biochemistry 1999 Sep. 14;38(37):12041-51).

A related embodiment of the invention is a method of increasing the stability of insulin polypeptides in a solution comprising combining the insulin polypeptides with a compound having the general formula A¹—L¹—S—L²—A², wherein the amount of compound in the solution is sufficient to inhibit the aggregation of the insulin polypeptides in the solution. Another related embodiment of the invention is method of producing a stabilized insulin solution comprising combining insulin with a compound having the general formula A¹—L¹—S—L²—A², wherein the compound inhibits the aggregation of the insulin in the solution.

Another embodiment of the invention is a method of making a compound for use in inhibiting the aggregation of insulin polypeptides in solution comprising a first step of constructing a compound having the general formula A¹—L¹—S—L²—A², wherein S comprises a molecular scaffold designed to have from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are carboxylic acid groups. Once this compound is constructed, the second step of the method entails testing the compound in an assay of insulin polypeptide aggregation, wherein the level of insulin polypeptide aggregation that is observed in a solution comprising insulin polypeptides and the compound is less than that observed in a control sample to which no compound has been added. A related embodiment of the invention is a method of identifying a compound for use in inhibiting the aggregation of insulin polypeptides in solution comprising combining insulin polypeptides with a compound having the general formula A¹—L¹—S—L²—A² and then characterizing the compound in an assay of insulin polypeptide aggregation, wherein the level of insulin polypeptide aggregation that is observed in the solution comprising the insulin polypeptides and the compound is less than that observed in a control sample to which no compound has been added. In such methods, the inhibition of aggregation is typically observed in a spectrophotometric assay of turbidity, a Thioflavin-T assay (e.g. as described in Example 1 below) or laser-induced light scattering.

The compound in the methods described above have a number of specific preferred embodiments. For example, in preferred embodiments, S has the structure:

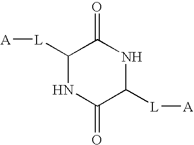

In certain preferred embodiments, wherein L¹ and L² have the structure:

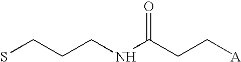

In addition, In certain preferred embodiments A¹ and A² have the structure:

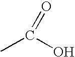

In typical embodiments of the invention, wherein the compound comprises a compound having a general formula shown in FIG. 2. For example, in an illustrative preferred embodiment, the compound has the general formula:

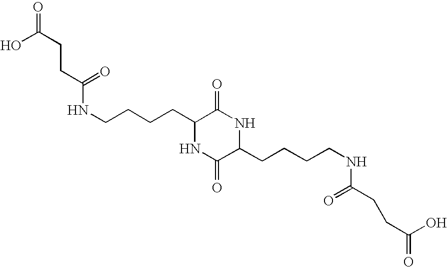

The formulations of the invention disclosed herein have a number of preferred embodiments. For example, because monomeric insulin analog formulations have a propensity to aggregate and to become unstable when exposed to thermo-mechanical stress, the methods and formulations described herein are particularly useful for addressing this characteristic of these analogs. For example, in one such preferred embodiment, the insulin polypeptide is a monomeric insulin analog formulations such as LISPRO insulin. As is described herein, the various formulations of the invention can comprise a wide variety of molecules typically used in this art. For example, certain preferred embodiments of the invention are those in which the formulation includes zinc. In addition, the various formulations are preferably pharmaceutical compositions (e.g. prepared in a pharmaceutically acceptable carrier).

Yet another embodiment of the invention is a method of inhibiting the generation of an immune response to exogenous insulin comprising administering the exogenous insulin in a solution combination with a compound having the general formula A¹—L¹—S—L²—A² wherein S comprises from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are carboxylic acid groups, wherein the amount of compound combined with the insulin polypeptides is sufficient to inhibit the aggregation of the insulin polypeptides in the solution. A related embodiment of the invention is a method of inhibiting the formation of immunoreactive insulin complexes in a solution comprising combining insulin in a solution a compound having the general formula A¹—L¹—S—L²—A², wherein the amount of compound combined with the insulin polypeptides is sufficient to inhibit the aggregation of the insulin polypeptides in the solution.

For use in the applications described or suggested above, kits are also provided. Such kits may comprise a carrier means being compartmentalized to receive in close confinement one or more container means such as vials, tubes, and the like, each of the container means comprising one of the separate elements to be used in the method. For example, one of the container means may comprise a compounds as described above for use in the methods disclosed herein. Such kits of the invention will typically comprise the container described above and one or more other containers comprising materials desirable from a commercial and user standpoint, including buffets, diluents, tubing, filters, needles, syringes, and package inserts with instructions for use. A label may be present on the container to indicate that the composition is used for a specific therapy or non-therapeutic application, and may also indicate directions for either in vivo or in vitro use, such as those described herein.

The invention disclosed herein is readily adapted for use with the wide variety of insulin formulations known in the art. Typical regular insulin formulations include insulin in an un-buffered or phosphate buffered solution containing glycerin for isotonicity, zinc ions for stability and a phenolic preservative such as phenol or m-cresol. These formulations are not sufficiently stable, either chemically or physically, for use in implantable pumps. In the history of implantable pump therapy (1979 to present) there has been only one commercially acceptable formulation due to the original work of Thurow and Geissen and expanded by Grau (for typical related art, see e.g. U.S. Pat. Nos. 4,608,364, 4,701,440, 4,801,684, 4,783,441 and 4,885,164). This formulation is described in U.S. Pat. No. 4,783,441 to Thurow et al., assigned to Hoechst (now Aventis) and is similar to the regular insulin formulations with the exception that the buffer system is Tris (tris-hydroxymethyl amino methane) and with the addition of Genapol, a polyoxyethylene, polyoxypropylene copolymer non-ionic surfactant. It has been speculated that the Genapol surfactant either surrounds the insulin molecule in solution thus guaranteeing a hydrophilic environment for the insulin, or that the Genapol coats the surfaces of the device limiting the potential for insulin monomer to denature on the surface.

Typical insulin analog formulations include insulin analog in an un-buffered or phosphate buffered solution containing glycerin for isotonicity, zinc ions for stability and a phenolic preservative such as phenol or m-cresol. Insulin LISPRO is an analog of human insulin where the B28 Proline and B29 Lysine are interchanged. The effects of this change are to fundamentally decrease the propensity to form the hexameric insulin structure and to increase the relative amount of insulin monomer present in solution. DeFelippis and colleagues in U.S. Pat. No. 6,034,054 describe a formulation of LISPRO insulin analog that shows remarkable physical stability when measured in-vitro. This formulation is similar to the implantable insulin formulation described by Thurow et al. in U.S. Pat. No. 4,783,441, however the Tris concentration is three fold lower and there is no Genapol in the formulation. Interestingly, addition of Genapol to the insulin LISPRO formulation greatly decreases the in-vitro physical stability suggesting that there is a different mechanism for aggregation in LISPRO formulations than in regular insulin formulations. Moreover, LISPRO formulations are remarkably physically stable when tested in-vitro in a standard physical insult model compared to regular human insulin formulations and the LISPRO preparation shows almost identical chemical stability compared to regular insulin. In spite of these findings, the in-vivo and in-vitro stability of the LISPRO in a catheter occlusion model in the presence of CO₂ is markedly less than the regular human insulin formulations.

Unfortunately, the in-vivo data suggest that the very physically stable LISPRO formulations described by DeFilippis et al. in U.S. Pat. No. 6,034,054 are not acceptable in-vivo. Specifically, in a series of parallel experiments using a diabetic canine model, implantable pumps were used with either LISPRO formulation described in U.S. Pat. No. 6,034,054 or the regular insulin formulations described in U.S. Pat. No. 4,783,441. While the regular insulin formulations showed the expected 90-day stability, catheter blockages were observed with the LISPRO formulations in only a few weeks. With the LISPRO formulations, the catheters were blocked with material that was identified by mass spectroscopy as a mixture of insulin and Tris in a 10/90 ratio. This surprising finding suggests a potential mechanism of LISPRO precipitation followed by LISPRO-Tris interaction coupled with reaction of CO₂ with the Tris to form the di-Tris carbamide.

The insulin compositions of the invention are preferably in a carrier; preferably a pharmaceutically-acceptable carrier. Suitable carriers and their formulations are described in Remington's Pharmaceutical Sciences, 16th ed., 1980, Mack Publishing Co., edited by Oslo et al. Typically, an appropriate amount of a pharmaceutically-acceptable salt is used in the formulation to render the formulation isotonic. Examples of the carrier include saline, Ringer's solution and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of insulin composition being administered.

The concentration of the insulin species in the present formulations typically ranges from 1.2 mg/ml to 50 mg/ml. A preferred range of insulin concentration is from about 3.0 mg/ml to about 35 mg/ml. More preferred concentrations are about 3.5 mg/ml, about 7 mg/ml, about 14 mg/ml, about 17.5 mg/ml, and about 35 mg/ml which correspond approximately to formulations having about 100 units, about 200 units, about 400 units, about 500 units, and about 1000 units of insulin activity per ml, respectively.

The concentration of zinc in the formulations ranges from about 0.5 μg/ml to about 370 μg/ml, and should be such that at least two zinc atoms are available to complex with the six insulin molecules in each hexamer. The ratio of total zinc (complexed zinc plus uncomplexed zinc) to insulin analog hexamer should be between 2 and 4. A ratio of about 3 to about 4 atoms of total zinc per insulin analog hexamer complex is preferred.

The minimum concentration of phenolic preservative that is required to form the monomeric insulin analog hexamer in the present formulations. For some purposes, such as to meet compendial preservative effectiveness requirements for multi-use formulations, the concentration of phenolic preservative in the present formulations may be increased above that required to form hexamers to an amount necessary to maintain preservative effectiveness. The concentration of preservative necessary for effective preservation depends on the preservative used, the pH of the formulation, and whether substances that bind or sequester the preservative are also present. Generally, the amount necessary can be found in, e.g., WALLHAUSER, K. D H., DEVELOP. BIOL. STANDARD. 24, pp. 9-28 (Basel, S. Krager, 1974). When formulated, the insulin analog hexamer complex used in the present formulation binds as many as seven phenolics, though generally, only six phenolics are bound to the hexamer. A minimum of about three phenolics is requited for hexamer formation. When preservative is required for antimicrobial effectiveness, the preferred phenolic concentration is about 23 mM to about 35 mM. M-cresol and phenol, either separately or in mixtures, are preferred preservatives.

The formulations may optionally contain an isotonicity agent. The formulations preferably contain an isotonicity agent, and glycerin is the most preferred isotonicity agent. The concentration of glycerin, when it is used, is in the range known in the art for insulin formulations, preferably about 16 mg/ml.

In addition to utilizing a compound having the general formula: A¹—L¹—S—L²—A² to inhibit the aggregation of insulin polypeptides, the utilization of additional stabilizers typically used in the art are also contemplated for use in the formulations described herein. For example, methionine is included in the disclosed pharmaceutical formulations as a means to effectively inhibit the oxidation of methionine residues in the protein. In addition, nonionic surfactants such as polysorbate 80 may be included to inhibit the damage to polypeptides that can occur with freeze-thawing and mechanical shearing. Moreover, EDTA and other known scavengers of metal ions (which are known to catalyze many oxidation reactions), may be added to further stabilize the compositions.

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) may optionally be added to the formulation. These additives are not required to achieve the great advantage of the present invention, but may be useful if the formulations will contact plastic materials.

Administration may be via any route known to be effective by the physician of ordinary skill. Parenteral administration is preferred. Parenteral administration is commonly understood as administration by other than a gastro-intestinal route. Preferred parenteral routes for administering the formulations of the present invention include intravenous, intramuscular, subcutaneous, intraperitoneal, intraarterial, nasal, pulmonary, and buccal routes. Intravenous, intraperitoneal, intramuscular, and subcutaneous routes of administration of the compounds used in the present invention are more preferred parenteral routes of administration. Intravenous, intraperitoneal, and subcutaneous routes of administration of the formulations of the present invention yet more highly preferred.

Administration via certain parenteral routes may involve introducing the formulations of the present invention into the body of a patient through a needle or a catheter, propelled by a sterile syringe or some other mechanical device such as an continuous infusion system. A formulation provided by the present invention may be administered using a syringe, injector, pump, or any other device recognized in the art for parenteral administration. A formulation of the present invention may also be administered as an aerosol for absorption in the lung or nasal cavity. The formulations may also be administered for absorption through the mucus membranes, such as in buccal administration.

The amount of a formulation of the present invention that is administered to treat a pathology such as diabetes or hyperglycemia depends on a number of factors, among which are included, without limitation, the patient's sex, weight and age, the underlying causes of the condition or disease to be treated, the route of administration and bioavailability, the persistence of the administered monomeric insulin analog in the body, the formulation, and the potency of the monomeric insulin analog. Where administration is intermittent, the amount per administration should also take into account the interval between doses, and the bioavailability of the monomeric insulin analog from the formulation. Administration of the formulation of the present invention could be continuous. It is within the skill of the ordinary physician to titrate the dose and infusion rate or frequency of administration of the formulation of the present invention to achieve the desired clinical result.

Monomeric insulin analogs used in the present invention can be prepared by any of a variety of recognized peptide synthesis techniques including classical solution methods, solid phase methods, semi-synthetic methods, and recombinant DNA methods. Chance, et al., U.S. Pat. No. 5,514,646, issued May 7, 1996, discloses the preparation of various monomeric insulin analogs with sufficient detail to enable one skilled in the art to prepare any of the monomeric insulin analogs used in the present invention.

Both zinc and a phenolic preservative are used to achieve a complex that is stable and capable of rapid dissociation and onset of action. The hexamer complex consists of two zinc ions per hexamer of human insulin analog, and at least three molecules of a phenolic preservative selected from the group consisting of chlorocresol, m-cresol, phenol, and mixtures thereof. Soluble monomeric insulin analog is converted to the hexamer complex by dissolving the monomeric insulin analog in a diluent containing the phenolic preservative in suitable quantities at a pH of about 7 to about 8 and then adding zinc. Zinc is preferably added as a zinc salt, such as, without limitation, zinc acetate, zinc bromide, zinc chloride, zinc fluoride, zinc iodide, and zinc sulfate. The skilled artisan will recognize that there are many other zinc salts which also might be used to make the monomeric insulin analog complexes that are part of the present invention. Preferably, zinc acetate, zinc oxide, or zinc chloride is used because these compounds do not add new chemical ions to commercially accepted processes.

Dissolution of the monomeric insulin analog may be aided by what is commonly known as “acid dissolution.” For acid dissolution, the pH the aqueous solvent is lowered to about 3.0 to 3.5 with a physiologically tolerated acid, preferably HCl, to aid in the dissolution of the monomeric analog. Other physiologically tolerated acids include, without limitation, acetic acid, citric acid, and sulfuric acid. Phosphoric acid is preferably not used to adjust pH in preparing the formulations of the present invention. The pH is then adjusted with a physiologically tolerated base, preferably sodium hydroxide, to about pH 7.3 to 7.5. Other physiologically tolerated bases include, without limitation, potassium hydroxide and ammonium hydroxide. Thereafter, the phenolic preservative and zinc are added.

Parenteral formulations of the present invention can be prepared using conventional dissolution and mixing procedures. To prepare a typical formulation for example, a measured amount of insulin and insulin analog is combined with the desired preservative, a zinc compound, and the buffering agent, in water in sufficient quantities. The formulation is generally sterile filtered prior to administration. Variations of this process would be recognized by one of ordinary skill in the art. For example, the order the components are added, the order in which pH is adjusted, if any, the temperature and ionic strength at which the formulation is prepared, may be optimized for the concentration and means of administration used.

As disclosed herein, the relative ability of various compositions having the general formula: A¹—L¹—S—L²—A² to inhibit the aggregation of insulin polypeptides can be determined by a number of methods known in the art. Preferably the inhibition of insulin polypeptide aggregation is determined by a spectrophotometric assay of turbidity, an assay with Thioflavin-T (as illustrated, for example in Example 1) or laser-induced light scattering.

There are a number of procedures known in the art to be useful in evaluating the stability of insulin compositions. Such methods include spectrophotometric measurements of dynamic light scattering, gel permeation chromatography, near- and far-ultraviolet circular dichroism etc. (see, e.g. Baudys et al., J Pharm Sci 1995 January;84(1): 28-33; Bugamelli et al., Arch Pharm (Weinheim) 1998 April; 331(4): 133-138; and Bremas et al., Biochemistry 1990 2;29(39): 9289-9293).

Typical methods for determining insulin stability are provided in Example 1 below. Briefly, these methods include two phases. The first phase includes the following steps. Preparing a statistically relevant number of identical samples of a protein formulation to yield a one or more sample types, where the protein is susceptible to changes in its native conformation yielding non-native conformers of the protein. A small molecular agent or probe that yields a change upon binding to a non-native conformer of the protein is then added to the samples. Preferably the probe is Thioflavin-T. A controlled stress is then applied to all sample types, where the controlled stress applied causes the protein to exhibit a change in its native conformation. The sample types are then monitored to yield time-dependent data that are related to a degree of protein conformational change for each sample type. The second phase includes applying a survival analysis to the data obtained for each sample type and comparing the survival analysis for each sample type to determine the relative physical stability of the protein formulations under evaluation. A preferred controlled stress suitable for use in embodiments of the invention is agitation. A preferred method to monitor the change in protein conformation is via fluorescence. An example of a protein conformational change suitable for use in the invention is the change in the physical structure of insulin from its native conformation to the fibril form of insulin.

Other embodiments of the invention consists of methods for treating diabetes consisting of administering an effective dose of the above-mentioned formulations to a patient in need thereof. A related embodiment of the invention consists of a method for treating hyperglycemia consisting of administering an effective dose of the disclosed formulation a patient in need thereof. In a preferred embodiments of these methods, the formulation is administered using a continuous infusion system.

Illustrative Compounds of the Invention

As is shown in FIGS. 2 and 3 for example, a variety of compounds having the general formula A¹—L¹—S—L²—A² can be used in the methods and formulations described herein. An illustrative example of such compounds are those wherein the scaffold subunit comprises a diketopiperazine. Diketopiperazines are known to be used in certain aspects of insulin purification as are described, for example, in U.S. Pat. Nos. 6,071,497 and 6,444,226, the contents of which are incorporated herein in their entirety. For purposes of clarity, a brief discussion of such typical molecules and procedures for their synthesis is provided in the following section.

Diketopiperazines or their substitution analogs are rigid planar rings with at least six ring atoms containing heteroatoms and unbonded electron pairs. One or both of the nitrogens can be replaced with oxygen to create the substitution analogs diketomorpholine and diketodioxane, respectively. The general formula for a compound having the general formula A¹—L¹—S—L²—A² wherein the scaffold subunit consists of a diketopiperazine is shown below.

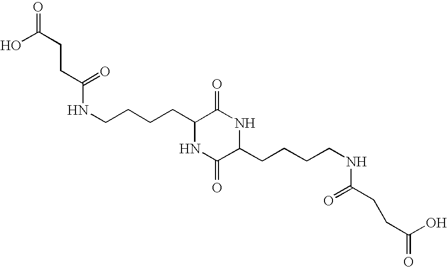

As is known in the art, various functional derivatives of this molecule can be synthesized, for example the side chains can be further functionalized with an alkene or alkyne group at any position, one or more of the carbons on the side chain can be replaced with an oxygen, for example, to provide short polyethylene glycol chains, one or more of the carbons can be functionalized with an acidic or basic group, as described above, and wherein the ring atoms are either O or N. Examples of acidic side chains include; but are not limited, to cis and trans —CH═CH—CO₂H, —CH(CH₃)═CH(CH₃)—CO₂H, —(CH₂)₃—CO₂H, —CH₂CH(CH₃)—CO₂H, —CH(CH₂CO₂H)═CH₂, -(tetrafluoro)benzoic acid, -benzoic acid and —CH(NHC(O)CF₃)—CH₂—CO₂H.

Examples of basic side chains include, but are not limited to, -aniline, -phenyl-C(NH)NH₂, -phenyl-C(NH)NH(alkyl), -phenyl-C(NH)N(alkyl)₂ and —(CH₂)₄NHC(O)CH(NH₂)CH(NH₂)CO₂H. Examples of zwitterionic side chains include, but are not limited to, —CH(NH₂)—CH₂—CO₂H and —NH(CH₂)₁₂CO₂H.

The term aralkyl refers to an aryl group with an alkyl substituent. The term heterocyclic-alkyl refers to a heterocyclic group with an alkyl substituent. The term alkaryl refers to an alkyl group that has an aryl substituent. The term alkyl-heterocyclic refers to an alkyl group that has a heterocyclic substituent. The term alkene, as referred to herein, and unless otherwise specified, refers to an alkene group of C₂ to C₁₀, and specifically includes vinyl and allyl. The term alkyne, as referred to herein, and unless otherwise specified, refers to an alkyne group of C₂ to C₁₀. As used herein, “diketopiperazines” includes diketopiperazines and derivatives and modifications thereof falling within the scope of the general formula (wherein X is preferably nitrogen or oxygen):

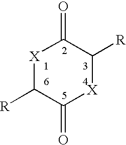

Diketopiperazines can be synthesized by one of a variety of methods known in the art. For example, diketopiperazines can be formed by cyclodimerization of amino acid ester derivatives, as described by Katchalski, et al., J. Amer. Chem. Soc. 68:879-80 (1946), by cyclization of dipeptide ester derivatives, or by thermal dehydration of amino acid derivatives in high-boiling solvents, as described by Kopple, et al., J. Org. Chem. 33(2):862-64 (1968), the teachings of which are incorporated herein by reference. 2,5-diketo-3,6-di(aminobutyl)piperazine (Katchalski et al. refer to this as lysine anhydride) was prepared via cyclodimerization of N-epsilon-P-L-lysine in molten phenol, similar to the Kopple method in J. Org Chem., followed by removal of the blocking (P)-groups with 4.3 M HBr in acetic acid. This route is preferred because it uses a commercially available starting material, it involves reaction conditions that are reported to preserve stereochemistry of the starting materials in the product and all steps can be easily scaled up for manufacture.

Diketomorpholine and diketooxetane derivatives can be prepared by stepwise cyclization in a manner similar to that disclosed in Katchalski, et al., J. Amer. Chem. Soc. 68:879-80 (1946). In addition, diketopiperazines can be radiolabeled. Means for attaching radiolabels are known to those skilled in the art. Radiolabeled diketopiperazines can be prepared, for example, by reacting tritium gas with those compounds listed above that contain a double or triple bond. A carbon-14 radiolabeled carbon can be incorporated into the side chain by using ¹⁴C labeled precursors which are readily available. These radiolabeled diketopiperazines can be detected in vivo after the resulting microparticles are administered to a subject. In preferred embodiments of the invention, the molecules having the general formula A¹—L¹—S—L²—A² are symmetrical. For example, diketopiperazine derivatives are symmetrical when both side chains are identical. The side chains typically include a carboxylic acid moieties as shown in FIGS. 2 and 3.

One example of a symmetrical diketopiperazine derivative is 2,5-diketo-3,6-di(4-succinylaminobutyl)piperazine. 2,5-diketo-3,6-di(aminobutyl) piperazine is exhaustively succinylated with succinic anhydride in mildly alkaline aqueous solution to yield a product which is readily soluble in weakly alkaline aqueous solution, but which is quite insoluble in acidic aqueous solutions. When concentrated solutions of the compound in weakly alkaline media are rapidly acidified under appropriate conditions, the material separates from the solution as microparticle. Other preferred compounds can be obtained by replacing the succinyl group(s) with glutaryl, maleyl or fumaryl groups.

Asymmetrical molecules having the general formula A¹—L¹—S—L²—A² are also contemplated. One method for preparing unsymmetrical diketopiperazine derivatives is to protect functional groups on the side chain, selectively deprotect one of the side chains, react the deprotected functional group to form a first side chain, deprotect the second functional group, and react the deprotected functional group to form a second side chain. Diketopiperazine derivatives with protected acidic side chains, such as cyclo-Lys(P)Lys(P), wherein P is a benzyloxycarbonyl group, or other protecting group known to those skilled in the art, can be selectively deprotected. The protecting groups can be selectively cleaved by using limiting reagents, such as HBr in the case of the benzyloxycarbonyl group, or fluoride ion in the case of silicon protecting groups, and by using controlled time intervals. In this manner, reaction mixtures which contain unprotected, monoprotected and di-protected diketopiperazine derivatives can be obtained. These compounds have different solubilities in various solvents and pH ranges, and can be separated by selective precipitation and removal. An appropriate solvent, for example, ether, can then be added to such reaction mixtures to precipitate all of these materials together. This can stop the deprotection reaction before completion by removing the diketopiperazines from the reactants used to deprotect the protecting groups. By stirring the mixed precipitate with water, both the partially and completely reacted species can be dissolved as salts in the aqueous medium. The unreacted starting material can be removed by centrifugation or filtration. By adjusting the pH of the aqueous solution to a weakly alkaline condition, the asymmetric monoprotected product containing a single protecting group precipitates from the solution, leaving the completely deprotected material in solution.

By carefully adjusting the solution pH, the deprotected derivative can be removed by filtration, leaving the partially and totally deprotected derivatives in solution. By adjusting the pH of the solution to a slightly acidic condition, the monoprotected derivative precipitates out of solution and can be isolated. Zwitterionic diketopiperazine derivatives can also be selectively deprotected, as described above. In the last step, adjusting the pH to a slightly acidic condition precipitates the monoprotected compound with a free acidic group. Limited removal of protecting groups by other mechanisms, including but not limited to cleaving protecting groups that are cleaved by hydrogenation by using a limited amount of hydrogen gas in the presence of palladium catalysts. The resulting product is also an asymmetric partially deprotected diketopiperazine derivative. These derivatives can be isolated essentially as described above. The monoprotected diketopiperazine is reacted to produce a diketopiperazine with one sidechain and protecting group. Removal of protecting groups and coupling with other side chains yields unsymmetrically substituted diketopiperazines. Other materials that exhibit this response to pH can be obtained by functionalizing the amide ring nitrogens of the diketopiperazine ring.

The present invention is further detailed in the following Examples which are offered by way of illustration and are not intended to limit the invention in any manner. All patent and literature references cited in the present specification are hereby incorporated by reference in their entirety. For purposes of clarity, certain techniques that are well known and well described in the art are reproduced herein.

### EXAMPLES

### Example 1

### Assessing Aggregation of Insulin Compositions

As is known in the art, there are a wide variety of methods that can be used to evaluate the stability of insulin compositions including spectrophotometric measurements of dynamic light scattering, gel permeation chromatography, near- and far-ultraviolet circular dichroism etc. (see, e.g. Baudys et al., J Pharm Sci 1995 January;84(1): 28-33; Bugamelli et al., Arch Pharm (Weinhein) 1998 April; 331(4): 133-138; and Bremas et al., Biochemistry 1990 2;29(39): 9289-9293). For example, the relative stability of a formulation can be assessed by a comparative analyses of the properties of a control formulation (e.g. one consisting of insulin polypeptides in the absence of the compounds disclosed herein having the general formula A¹—L¹—S—L²—A² with a formulation under investigation (e.g. one consisting of insulin polypeptides in the presence of the compounds disclosed herein having the general formula A¹—L¹—S—L²—A²).

As disclosed herein, the conformational status (which is known to be related to a proteins stability) of insulin can also be evaluated with a spectroscopic agent or probe that preferentially binds to a non-native conformer of insulin (e.g. one exhibiting a non-native β-pleated sheet conformation). One example of a small molecular spectroscopic probe of protein structure is Thioflavin-T. Protocols are provide below as illustrative examples of methods that can be used to asses the physical stability of insulin compositions.

Typical embodiments of such methods involve placing a small amount of an insulin formulation to be evaluated under a controlled stress. This controlled stress is physically translated to the protein contained in a particular formulation. A comparison of two or more protein formulations that differ in composition then yields the relative physical stability of the proteins formulations under evaluation. In such assays, a typical sample volume is about 50 μl to about 500 μl, most preferably about 200 μl. The medium chosen for the analysis can be any medium in which the physical stability of a particular protein is desired to be evaluated, such as aqueous solutions, organic solvents, and the like.

A change in the physical state of a protein such as insulin, i.e., production of one or more non-native protein states, is detected spectroscopically using an spectroscopic probe that preferentially binds to a non-native form of the protein, as compared to its binding to the native form of the protein. The detection of this induced change in protein state, caused by the applied stress, can be observed by following a concomitant change in spectra of the spectroscopic probe upon its binding to a non-native state of the protein. This change in the spectra of the spectroscopic probe can be monitored by numerous spectral techniques, such as fluorescence, absorbance, nuclear magnetic resonance (NMR), circular dichroism (CD), or the like.

Other embodiments of the invention include monitoring a change in the physical state of protein by observing changes in the bulk physical properties of the protein formulations under evaluation. These techniques involve monitoring a change in shape and/or size of the protein as a function of the applied stress, including monitoring changes in the frictional properties, viscosity, turbidity, light scattering, or the like, of the protein formulations under evaluation. The use of these techniques in embodiments of the invention do not require the addition of a spectroscopic agent to probe the change in the conformational state of the protein in a given formulation.

The stress applied is preferably a controlled physical stress, including agitational, vibrational, rotational, shearing, ultrasonic stresses, or the like. Other types of applied stress are included in embodiments of the invention, such as thermal stress, photochemical stress, or the like. When applying a thermal stress, concomitant changes in the physical states of the protein result, however, thermal stress also may affect the chemical state of the protein. In embodiments of the invention utilizing a photochemical stress, generally changes in the physiochemical state of the protein is probed. Further, the controlled stress applied can be a combination of two or more stresses, such as agitation of the protein formulations of interest at elevated temperatures.

As noted above, the spectroscopic agent or probe is preferably a small molecule that preferentially binds to a non-native conformer of the protein. One example of a small molecular spectroscopic probe of protein structure is Thioflavin-T. Thioflavin-T is a fluorescent dye that has been widely used for the detection of amyloid fibrils. In the presence of fibrils, and perhaps other protein configurations as well, Thioflavin-T gives rise to a new excitation maximum at about 450 nm and enhanced emission at about 482 nm when bound to a fibril protein form. Unbound Thioflavin-T is essentially non-fluorescent at these wavelengths. Recently, Thioflavin-T has been used to elucidate the mechanism of fibril formation in insulin (see, e.g. Nielson, et al., Biochemistry, 2001, 40, p. 6036). Other small molecules can be used as probes of the changes in protein structure from native to non-native states. Examples of other small molecular, spectroscopic probes is the “exposed hydrophobic patch” probe and the “exposed coordination site” probe. A “hydrophobic patch” probe preferentially binds to exposed hydrophobic patches of a protein. These hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline, or the like. Other spectroscopic probes are metal-amino acid complexes, such as cobalt metal complexes of hydrophobic amino acids, such as phenylalanine, leucine, isoleucine, methionine, and valine, or the like. As is the case with Thioflavin-T, these small molecular, spectroscopic probes yield a spectroscopic change upon binding to a non-native form of the protein of interest, such as a change in fluorescence, a change in absorbance, a change in circular dichroism, and the like.

Another example of a small molecular probe, is a probe of the coordination saturation in non-native states of a metalloprotein. Examples of these molecular probes are spectroscopically active and unsaturated coordination metal complexes, such as ruthenium-pyridyl complexes, ruthenium-phenanthroline complexes, or the like. These spectroscopic probes bind to one or more exposed coordination sites in non-native conformations of a metalloprotein. These exposed coordination sites generally are bound to specific amino acid ligands in the native protein, but become open to coordination by the small molecular probe in non-native states of the metalloprotein.

Other spectroscopic systems utilizing spectroscopic probes also can be utilized in embodiments of the invention, such as fluorescence systems based on FRET (fluorescence resonance energy transfer) and PET (photo-induced electron transfer), such as those disclosed in U.S. Pat. No. 6,011,984, and the like, which is incorporated by reference in its entirety herein.

After gathering spectroscopic data reflective of the physical stability of one or more protein formulations, these data are compared using survival curve analyses. These survival curve analyses are statistical methods similar to those that have been used in the prior art, for example, in the analysis of data from clinical trials of a new pharmaceutical drug. In these clinical trials the survival of patients taking the drug is compared to the survival of patients taking a placebo or another drug. Thus, at the end of the trial, a percent survival is obtained for each patient population. As with any statistical method, the number of individuals, or samples, i.e., an N value, should be chosen to yield statistically significant results. In an embodiment of the invention that utilizes a 96 well microplate, as described below, the N value chosen is preferably about 16 to about 48 identical sample formulations with an N value of about 24 identical samples being most preferred. Other N values also may be used depending of the size of the microplate utilized, or other similar device for containing the samples.

In preferred embodiments of the invention, the survival curve analysis compares the rate of protein aggregation of a first sample type, containing a statistically relevant number of identical samples of a particular insulin formulation, to that of one or more samples types containing a statistically relevant number of identical samples of different insulin formulations. In particular embodiments of the invention, protein aggregation, or changes in protein conformation, is equated with non-survival of the protein.

A protein reference batch of known physical stability can be used to estimate the physical stability of particular unknown insulin formulations. A comparison to a reference protein batch controls for small changes in starting conditions that can affect the absolute rate of aggregation, as well as controlling drift in the light source intensity and detector sensitivity. These small changes and drifts can affect the absolute intensity measurements. Additionally, the possibility of dye bleaching by incident radiation can be addressed by using a protein reference batch. Thus, the use of a protein reference batch controls for random variables in the experimental protocol. However, a protein reference batch is not necessary to the evaluation of the relative physical stability of a series of protein formulations. In this experimental design, each protein formulation acts as a reference to the other protein formulations under evaluation. Thus, the relative physical stability of a series of protein formulations can be determined without the use of a protein reference batch.

In a preferred assay, the median survival time for particular protein samples to reach a state where 50% of the protein is in an aggregated state, or 50% of the protein has not survived, is chosen as an end point of the experimental run. This end point is preferred as a metric and appears to adequately represent the physical stability of a given protein formulation. In other embodiments of the invention, other points on the survival curve can be used as a metric of the physical stability of the protein formulations under evaluation. The median survival, however, appears to represent and coincide with the average physical stability of the sample formulations. Thus in embodiments of the invention, the longer the median survival, the greater is the physical stability of the protein formulations under evaluation.

A survival curve analysis of the change in physical state of a particular protein is highly preferable because direct evaluation of the physical stability of protein formulations is difficult, to nearly impossible, to obtain simply from a profile of protein aggregation versus time. There are two main reasons for this difficulty. The first reason is experimental. The raw data are very noisy mostly due to a stirring device, such as Teflon bead, being in the light path of the fluorometer, as described below. The second reason is that the kinetics of aggregation is not well understood. For insulin, it has been proposed that the process of aggregation is autocatalytic (see, e.g. Sluzky et al., Proceedings of the National Academy of Sciences, 1991, 88 p. 9377). The actual reaction mechanism of aggregation of insulin, which includes fibril formation, however, is largely unknown. Accordingly an analytical solution to the aggregation kinetic mechanism of insulin is also unknown.

The use of a survival curve analysis applied to the spectroscopic data of changes in protein states yields a simple method which compares a statistically relevant number of samples of a given formulation to a statistically relevant number of samples of another formulation, yielding a relative physical stability profile of the sample types under evaluation. Accordingly, no prior knowledge or understanding of the mechanism of aggregation, or the mechanism of protein conformational changes, is needed for applications of embodiments of the invention.

Once aggregation profiles are obtained for each sample type under evaluation, a calculation of the time to reach a certain fluorescence level is performed. This time to reach a certain fluorescence level is generally set at 50% survival, but can vary with the needs of particular experiments. This end point of the survival analysis represents a point in the aggregation profile that is at least beyond the initiation of aggregation. Moreover, the level of fluorescence obtained at the 50% survival point was determined to yield reliable results. This level of fluorescence is generally substantially greater than the background noise of the system and ensures that the results are statistically relevant. For experimental systems with greater or less noise, higher or lower levels of fluorescence can be set as end points.

The data typically consists of a series of times to the initiation of aggregation. Once the time to start aggregation is calculated, a standard Kaplan-Meier survival curve analysis (see, e.g. Campbell, M., and Machin, D., Medical Statistics, Wiley, New York, 1983. p. 112), where survival fractions are calculated as a function of time, is applied. For a comparison between the formulations of interest and a reference batch, for example, the log rank test equivalent to the Mantel-Haenszel test is performed. This test generates a P value testing the null hypothesis that the survival curves are identical.

Other important information that can be obtained from comparing two survival curves is their median survival and the ratio of the median survival of a reference batch, or other protein sample type, to the median survival of the sample type of interest. Median survival, as described above, is the time for 50% of the samples to reach a pre-determined level of fluorescence. If the samples do not show fluorescence at the end of the experiment, then median survival cannot be computed and we can only estimate a “minimum” median survival.

In the following typical protocol, the physical stability of particular insulin formulations are evaluated using a preferred embodiment of the accelerated physical stability method of the invention. However, the accelerated physical stability methods of the embodiments of the invention can be used to evaluate any protein that undergoes a change in conformation due to an application of a controlled stress.

A first step is to physically stress the insulin formulations under evaluation by controlled agitation. A series of identical insulin samples are prepared to yield a first sample type, or reference batch in this example, and another series of identical insulin samples are prepared to yield a second sample type. A small volume of each insulin sample is placed in an open well, i.e., exposed to air, of a 96-well microplate. One or more small, stirring device(s), preferably in the form of Teflon (polytetrafluoroethylene) bead(s), is also placed in each sample well. The Teflon bead has a very hydrophobic surface, which increases the interfacial tension within each well. However, the stirring device can be made of different materials, including hydrophilic materials Thioflavin-T, which is shown to bind to aggregated protein states is then added to each sample well (see, e.g. Levine, H., Protein Science, 1993, 2, p. 404).

Although this particular embodiment of the invention exposes the protein formulations to air, and thus increasing the interfacial tension, i.e., exposure to a air-water interfaces, other embodiments of the invention utilize placing the protein samples in sealed vials from which residual air is evacuated, thereby reducing the air-water interface. The physical stress applied is agitation of the microplate in a commercial instrument that also measures the fluorescence of Thioflavin-T as a function of the time of agitation. A typical instrument suitable for use in the accelerated physical stability methods of the invention is a Fluorskan fluorescence plate reader (Lab-systems). In this embodiment, the plate is orbitally agitated. However other forms of agitation, such as shaking and vibrating, are suitable for use in other embodiments of the invention. After a certain time of agitation, a curve is generated, which is a plot of aggregation, as measured by the increase in Thioflavin-T fluorescence intensity, as a function of time. The data generated are then subjected to a survival curve analysis.

A typical experiment begins by adding approximately 200 microliters of a given insulin formulation (e.g. a combination of insulin species to be examined) into multiple wells in the 96-well microplate. A single Teflon bead is added to the well together with an aliquot of Thioflavin-T dissolved in water. The microplate is covered with a Mylar sheet to reduce evaporation and avoid against accidental particulate contamination. The covered microplate is then placed in an incubated orbital shaker and is agitated with a controlled force. The preferred operational parameters are given in the Table 1.

TABLE 1
Operational Parameters for Protein Stability Estimation

Sample volume: 150-250 μl preferably 190 μl
Thioflavin-T: 10-30 μM, preferably 20 μM
Shaking speed: 480-1200 rpm, preferably 960 rpm
Shaking diameter: 1-5 mm, preferably 1 mm
Temperature: 25-40° C., preferably 37° C.
Microplate cover: 1-3 sheets of Mylar covers preferably one sheet.
Excitation wavelength: 440 nm-500 nm, preferably about 444 nm
Emission wavelength : 480 nm-520 nm, preferably about 510 nm
Measurement directions: top down
Number of wells per formulation: ≧24
Run time: 3-9 days or until more than 50% of
the samples start to aggregate

A representative snap shot of an aggregation profile for a single run using all 96 wells can then be taken. This snap shot gives the observer a quick look at the experimental results without any further analysis. A snap shot is taken at every measurement during the entire run. Once an aggregation profile is obtained, the time to reach a certain fluorescence level (e.g. 50) is calculated, which in this example represents the definite initiation of aggregation. The particular fluorescence level can be determined from a comparison to the background noise to ensure that the fluorescence level chosen is above the background noise. Once the time to start aggregation is calculated (e.g. t₅₀) one can plot a survival curve, where survival fractions are calculated using Kaplan-Meier method. These calculations are performed using commercially available software, such as Prism or an equivalent software.

### Example 2

### Synthesis of N-[2-(3-Carboxy-propionylamino)-ethyl]-succinamic acid

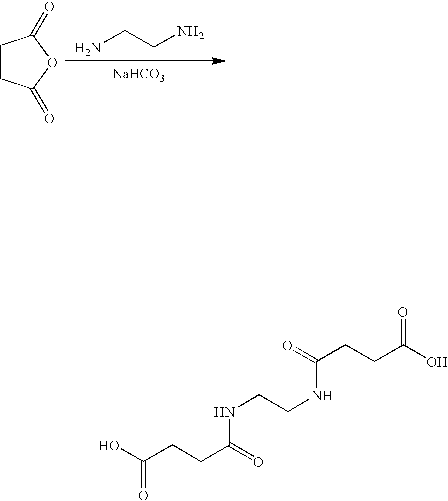

To a round bottom flask containing ethylene diamine (3.0 g, 50 mmol) dissolved in NaHCO₃ (sat'd, 200 mL) was added succinic anhydride (10 g, 100 mmol). CO₂ evolution occurred after ˜5 min. A white ppt formed after 20 min. The pH of the solution was made basic with NaOH (1N) causing the solution to turn clear, colorless. After 15 min, the solution was filtered. To the filtered solution was added citric acid (3.33 g, 17 mmol) and HCl (12N, 10 mL, 120 mmol) after which the reaction became cloudy due to formation of a white ppt. After 30 min, the ppt was filtered off to afford 5.01 g (39%) of a white solid. ¹H NMR (SOC₂D₆, 360 MHz) δ 2.26-2.30 (4H, m), 2.39-2.42 (4H, m), 3.04 (4H, d, J=2.4 Hz), 7.88 (2H, s).

C10H16N206

Exact Mass: 260.1008

Mol. Wt.: 260.2439

C, 46.15; H, 6.20; N, 10.76; O, 36.89.

### Example 3

cl Synthesis of N-[4-(3-Carboxy-propionylamino)-butyl]-succinamic acid

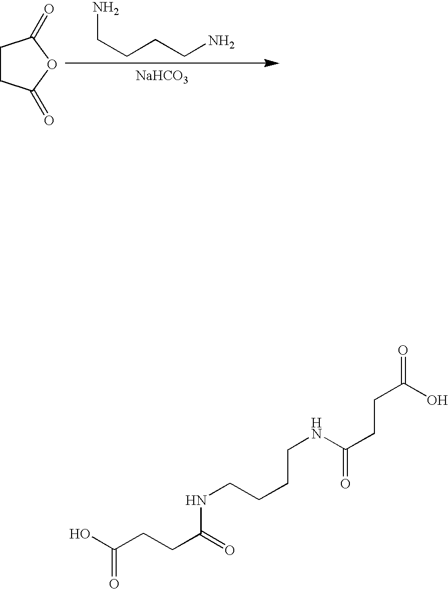

To a round bottom flask containing 1,4 diaminobutane (5.07 mL, 50 mmol) dissolved in NaHCO₃ (sat'd, 200 mL) was added succinic anhydride (10 g, 100 mmol). CO₂ evolution occurred after ˜5 min. A white ppt formed after 20 min. The pH of the solution was made basic with NaOH (1N) causing the solution to turn clear, colorless. After 15 min, the solution was filtered. To the filtered solution was added citric acid (3.33 g, 17 mmol) and HCl (12N, 10 mL, 120 mmol) after which reaction became cloudy due to formation of a white ppt. After 30 min, the ppt was filtered off to afford a white solid.

C12H20N206

Exact Mass: 288.1321

Mol. Wt.: 288.2971

C, 49.99; H, 6.99; N, 9.72; O, 33.30.

Modifications and variations of the present invention will be obvious to those of skill in the art from the foregoing detailed description. Such modifications and variations are intended to come within the scope of the following claims.

What is claimed is:

1. A method of inhibiting the aggregation of insulin polypeptides in a solution comprising combining the insulin polypeptides with a compound having the general formula: 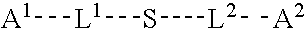
wherein S comprises from one to seven consecutively linked atoms selected from the group consisting of nitrogen, carbon, and oxygen, wherein at least one of the atoms is a carbon atom; L¹ and L² ate linking groups having from two to twelve consecutively linked atoms selected from the group consisting of nitrogen, carbon, oxygen, sulfur, and phosphorus; and A¹ and A² are organic acid moieties;
wherein the amount of compound combined with the insulin polypeptides is sufficient to inhibit the aggregation of the insulin polypeptides in the solution.

2. The method of claim 1, wherein the compound comprises a compound having a general formula shown in FIG. 2.

3. The method of claim 2, wherein the compound has the general formula:

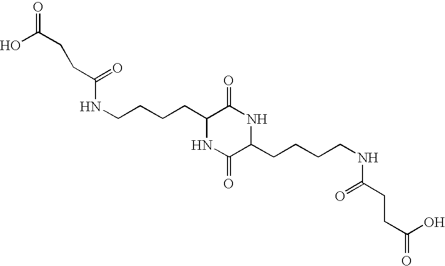

4. The method of claim 1, wherein S has the structure:

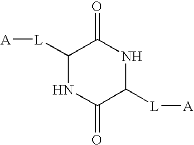

5. The method of claim 1, wherein L¹ and L² have the structure:

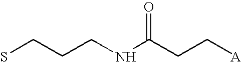

6. The method of claim 1, wherein A¹ and A² have the structure:

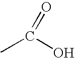

7. The composition of claim 1, wherein insulin polypeptide is LISPRO insulin.

8. The composition of claim 1, wherein the composition is a pharmaceutical composition.

9. The method of claim 1, wherein the solution comprises zinc.

10. The method of claim 1, wherein the compound further comprises a phenolic moiety.

11. The method of claim 1, wherein the inhibition of aggregation is observable in a spectrophotometric assay of turbidity or a Thioflavin-T assay.

12. A method of increasing the stability of insulin polypeptides in a solution comprising combining the insulin polypeptides with a compound having the general formula:
A¹—L¹—S—L²—A²
wherein S comprises from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² ate carboxylic acid groups; and wherein the amount of compound in the solution is sufficient to inhibit the aggregation of the insulin polypeptides in the solution.

13. The method of claim 12, wherein the compound has the general formula:

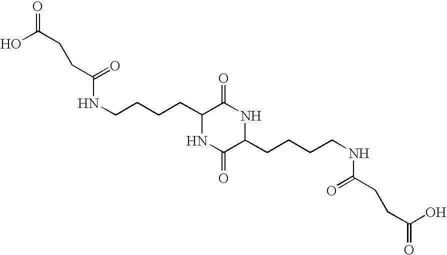

14. A method of producing a stabilized insulin solution comprising combining insulin with a compound having the general formula:
A¹—L¹—S—L²—A²
wherein S comprises from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are carboxylic acid groups; and wherein the compound inhibits the aggregation of the insulin in the solution.

15. The method of claim 14, wherein the compound has the general formula:

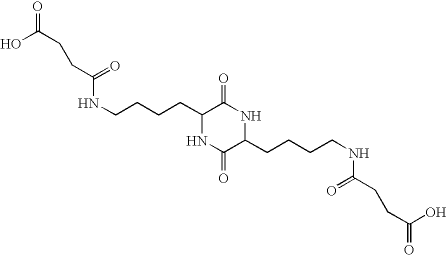

16. A method of making a compound for use in inhibiting the aggregation of insulin polypeptides in solution comprising:
(a) constructing a compound having the general formula:
A¹—L¹—S—L²—A²
wherein S comprises from one to seven atoms selected from the group consisting of nitrogen, carbon, and oxygen, wherein at least one of the atoms is a carbon atom; L¹ and L² ate linking groups having from two to twelve consecutively linked atoms selected from the group consisting of nitrogen, carbon, oxygen, sulfur, and phosphorus; and A¹ and A² are carboxylic acid groups; and
(b) testing the compound in an assay of insulin polypeptide aggregation, wherein the level of insulin polypeptide aggregation that is observed in a solution comprising insulin polypeptides and the compound is less than that observed in a control sample to which no compound has been added.

17. A method of identifying a compound for use in inhibiting the aggregation of insulin polypeptides in solution comprising combining insulin polypeptides with a compound having the general formula:
A¹—L¹—S—L²—A²
wherein S comprises from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are carboxylic acid groups; and
(b) characterizing the compound in an assay of insulin polypeptide aggregation, wherein the level of insulin polypeptide aggregation that is observed in the solution comprising the insulin polypeptides and the compound is less than that observed in a control sample to which no compound has been added.

18. A method of inhibiting the generation of an immune response to exogenous insulin comprising administering the exogenous insulin in a solution combination with a compound having the general formula:
A¹—L¹—S—L²—A²
wherein S comprises from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are carboxylic acid groups;
wherein the amount of compound combined with the insulin polypeptides is sufficient to inhibit the aggregation of the insulin polypeptides in the solution.

19. The method of claim 18, wherein the compound has the general formula:

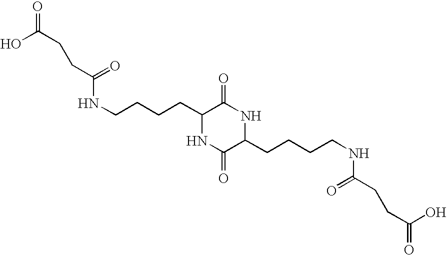

20. A method of inhibiting the formation of immunoreactive insulin complexes in a solution comprising combining insulin in a solution a compound having the general formula:
A¹—L¹—S—L²—A²
wherein S comprises from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are carboxylic acid groups;
wherein the amount of compound combined with the insulin polypeptides is sufficient to inhibit the aggregation of the insulin polypeptides in the solution.

21. The method of claim 20, wherein the compound has the general formula:

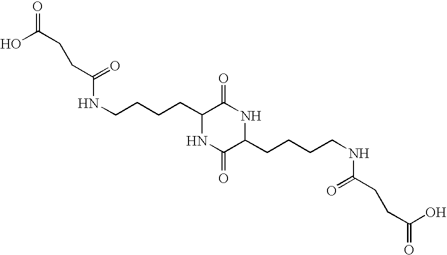

22. A method of inhibiting the aggregation of polypeptides in a solution comprising combining the polypeptides with a compound having the general formula:
A¹—L¹—S—L²—A²
wherein S comprises from one to seven atoms selected from the group consisting of nitrogen, carbon, and oxygen, wherein at least one of the atoms is a carbon atom; L¹ and L² are linking groups having from two to twelve consecutively linked atoms selected from the group consisting of nitrogen, carbon, oxygen, sulfur, and phosphorus; and A¹ and A² are carboxylic acid groups;
wherein the amount of compound combined with the polypeptides is sufficient to inhibit the aggregation of the polypeptides in the solution.

23. The method of claim 22, wherein the polypeptide is LISPRO insulin.

24. The method of claim 22, wherein the compound has the general formula:

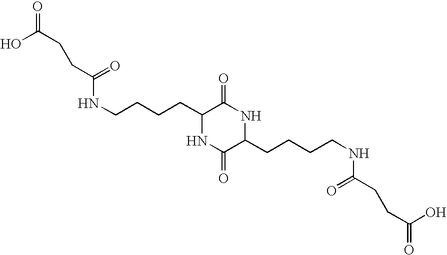

25. A compound having a general formula shown in FIG. 2.
